(19) United States
(12) Patent Application Publication
(10) Pub. No.: US 2026/0060776 A1
(43) Pub. Date: Mar. 5, 2026

(54) ADAPTIVE FUDICIALS FOR MULTI-MODAL RADIOTHERAPY

(71) Applicant: IZI Medical Products, LLC, Owings Mills, MD (US)

(72) Inventor: Kevin REYNOLDS, Owings Mills, MD (US)

(73) Assignee: IZI Medical Products, LLC, Owings Mills, MD (US)

(21) Appl. No.: 19/310,148

(22) Filed: Aug. 26, 2025

Related U.S. Application Data
(60) Provisional application No. 63/687,427, filed on Aug. 27, 2024.

(51) Int. Cl. A61B 90/00

(52) U.S. Cl. CPC A61B 90/39; A61B 2090/3908; A61B 2090/3966; A61B 2090/3987

(57) ABSTRACT

The present disclosure is related to an interstitial marker configured to be imaged when inserted into soft tissue of an organ, tumor or tumor bed within a subject's body, wherein the interstitial marker is optimized for multi-modality compatibility by maintaining necessary dimensions for visibility, trackability and material reduction in a beam path via customized pitch values to minimize proton beam perturbation.

### CROSS-REFERENCE TO RELATED APPLICATIONS

This application claims benefit of priority to U.S. Provisional Patent Application No. 63/687,427, entitled “ADAPTIVE FIDUCIALS FOR MULTI-MODAL RADIOTHERAPY,” filed Aug. 27, 2024. The entire contents and disclosures of these patent applications are incorporated herein by reference in their entirety.

This application refers to U.S. patent application Ser. No. 13/212,293 entitled, “ELONGATED MARKER FOR SOFT TISSUE VOLUME IDENTIFICATION,” filed Aug. 18, 2011, and U.S. patent application Ser. No. 10/284,037 entitled, “ELONGATED MARKER FOR SOFT TISSUE VOLUME IDENTIFICATION,” filed Oct. 29, 2002. The entire contents and disclosures of these patent applications are incorporated herein by reference.

### BACKGROUND

### Field of the Invention

The present disclosure relates generally to Image-guided radiotherapy (IGRT). More specifically, the present disclosure relates to fiducial markers that perform effectively across multiple radiotherapy modalities.

### Background of the Invention

Image-guided radiotherapy (IGRT) has become a critical advancement in modern radiation oncology, enabling improved accuracy in patient positioning and tumor localization. By incorporating imaging into the treatment process, IGRT allows for reduced treatment margins and increased sparing of surrounding healthy tissue. Fiducial markers are widely used in IGRT as stable, radiopaque reference points that enable precise alignment of treatment beams with the intended target. Their utility is well established in various treatment sites, including prostate cancer, where anatomical motion and positional variability present significant challenges to treatment precision.

Facilities that offer both photon and proton therapy often seek to streamline clinical workflows by employing a single fiducial marker type across modalities. However, current fiducial technologies present inherent trade-offs between imaging visibility and the generation of imaging artifacts. This balance becomes particularly problematic in proton therapy, where high-density fiducial markers can cause proton beam perturbations. Such perturbations can lead to dose shadowing and underdosing in tissue distal to the marker, creating dosimetric uncertainties that may compromise treatment efficacy. These effects are of particular concern in highly conformal proton therapy plans for prostate cancer, where precise delivery and dosage is essential.

Various mitigation strategies have been attempted, including limiting the number of treatment fields or modifying beam angles to avoid direct interaction with fiducials. However, these approaches constrain treatment planning flexibility and may reduce the potential dosimetric advantages of proton therapy.

Recent advancements in fiducial marker technology—such as liquid fiducials and polymer-encapsulated markers—have sought to reduce proton beam perturbation while maintaining adequate imaging visibility. While these designs represent incremental improvements, they have not fully addressed the problem of developing a fiducial marker that: (1) provides consistent, high-contrast visibility across all imaging modalities used in both photon and proton IGRT; (2) produces minimal imaging artifacts; and (3) causes negligible perturbation to proton dose distribution.

Accordingly, there remains a need in the art for fiducial markers that perform effectively across multiple radiotherapy modalities, offering both high visibility and minimal impact on beam quality. Such an innovation would improve the precision and versatility of IGRT, particularly in facilities employing both photon and proton therapy.

### SUMMARY

According to first broad aspect, the present disclosure provides an interstitial marker configured to be imaged when inserted into soft tissue of an organ, tumor or tumor bed within a subject's body, wherein the interstitial marker is optimized for multi-modality compatibility by maintaining necessary dimensions for visibility, trackability and material reduction in a beam path via customized pitch values to minimize proton beam perturbation, having a pitch value in a range of about 0.25 mm to about 1.5 mm, wherein the interstitial marker has a diameter of at least 0.50 mm.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration showing: (a) a Rondo phantom utilized for clinical imaging assessments; (b) a wax phantom constructed for a proton beam perturbation study according to one embodiment of the present disclosure.

FIG. 2 is a schematic illustration showing proton beam perturbation evaluation for: (a) schematic of the wax phantom setup; (b) film calibration curves for triple channel film dosimetry; (c) image of an irradiated film; (d) a corresponding dose map showing the dose decrease under fiducial projections according to one embodiment of the present disclosure.

FIG. 3 illustrates a visibility assessment including: (a) CBCT image demonstrating the visibility of customized fiducials with varying pitch values; (b) A and B, KV/KV images confirming the fiducials' visibility in IGRT according to one embodiment of the present disclosure.

FIG. 4 illustrates fiducial tracking with a CyberKnife® System including: (a-g) Dynamic treatment simulation images showing successful tracking of each fiducial across different angles and positions, with trackability largely independent of pitch values according to one embodiment of the present disclosure.

FIG. 5 is a schematic illustration showing a summary of fiducial tracking uncertainties under various imaging settings according to one embodiment of the present disclosure.

FIG. 6 graphically illustrates proton beam perturbation including: (a) the dose at each fiducial projection position (Fiducial 1-8, solid lines) as a function of depth distal to the fiducials. The average background dose at each depth is shown as the dashed line; (b) the percentage change in dose across each fiducial projection position relative to the surrounding background area as a function of depth distal to the fiducials according to one embodiment of the present disclosure.

FIG. 7 graphically illustrates dose (Gy) versus film depth (mm) according to one embodiment of the present disclosure.

FIG. 8 graphically illustrates dose perturvation (%) versus distance (mm) according to one embodiment of the present disclosure.

FIG. 9 is a radiological image illustrating a “starburst” image according to one embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing FIGS. may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For the purposes of the present disclosure, the term “adaptive fiducials (e.g., for multi-modal radiotherapy)” refers to marker-based systems that help improve the accuracy of radiation therapy by adapting to changes in a patient's anatomy or tumor position during treatment. These fiducials are particularly useful in image-guided radiation therapy (IGRT) and multi-modal treatments that combine different imaging and therapeutic techniques. Key features of adaptive fiducials in radiotherapy may include: high visibility across modalities—they are designed to be detectable in multiple imaging techniques like CT, MRI, PET, and fluoroscopy; real-rime tracking—they enable motion monitoring (e.g., due to breathing) and allow for adjustments during treatment; adaptive positioning—these fiducials adjust for anatomical changes, improving treatment precision over time; minimizing dosimetric errors—they help reduce radiation dose inaccuracies by ensuring proper alignment between treatment plans and the actual tumor position. Applications in multi-modal radiotherapy may include: prostate cancer—used to track prostate movement during external beam radiation therapy (EBRT); lung & liver cancer—helps account for organ motion due to breathing; stereotactic radiotherapy (SBRT/SRS)—ensures high precision for small tumors treated with high-dose radiation; MRI-Guided Radiotherapy—special fiducials compatible with MRI (non-metallic) improve soft-tissue visualization. Types of adaptive fiducials may include: gold markers (for X-ray/CT-guided therapy); biodegradable markers (absorb overtime, reducing long-term complications); electromagnetic (EM) fiducials (for real-time motion tracking); and hydrogel-based fiducials (MRI-compatible, non-metallic).

For the purposes of the present disclosure, the term “beam perturbation” refers to any change in the characteristics of a radiation beam (in radiation therapy—such as its intensity, shape, or distribution—caused by something the beam encounters before reaching its target. In the context of radiation therapy, when delivering X-rays, electrons, or protons, the beam can be disturbed by: patient-specific devices (immobilization masks, bite blocks, bolus material); fiducial markers (especially metallic ones); implants (dental fillings, pacemakers, surgical clips); beam modifiers (multi-leaf collimators, wedges, compensators); patient anatomy changes (air pockets, swelling, tissue density shifts). Types of perturbations may include: attenuation (the beam loses intensity as it passes through materials); scattering (beam particles are deflected, broadening the beam or changing its dose distribution); dose shadowing (regions behind a dense object receive less dose than planned); hot spots (scattering or secondary radiation causes unexpected higher doses in nearby tissues). Factors of importance may include: accuracy—radiation plans are based on predicted beam paths; perturbations can cause underdosing of tumors or overdosing of healthy tissue; planning adjustments—modern treatment planning systems account for known perturbations, but unexpected ones (like moving air cavities) can require replanning; special concern in proton therapy—protons are more sensitive to material changes because their range is so specific; even a few millimeters of density change can shift the Bragg peak.

For the purposes of the present disclosure, the term “Bragg peak” refers to a phenomenon in particle physics—and a key advantage of proton therapy—where charged particles like protons deposit most of their energy at a specific depth in tissue, just before coming to a stop.

For the purposes of the present disclosure, the term “CBCT” refers to cone beam computed tomography (or CBCT, also referred to as C-arm CT, cone beam volume CT, flat panel CT or Digital Volume Tomography (DVT)). CBCT is a medical imaging technique consisting of X-ray computed tomography where the X-rays are divergent, forming a cone.

For the purposes of the present disclosure, the term “CT” refers to computed tomography. It is a medical imaging technique that uses X-rays to create detailed, three-dimensional images of internal organs and tissues.

For the purposes of the present disclosure, the term “CyberKnife®” refers to a robotic radiosurgery system used to deliver highly precise radiation therapy to tumors and other medical conditions—without surgery. CyberKnife® employs focused beams of radiation to destroy tumor cells, guided by advanced imaging and robotics. Key Features may utilize a robotic arm to move around the patient to aim the radiation from hundreds of angles. This flexibility allows for highly conformal dose shaping around irregular tumors. Image Guidance may use real-time X-ray imaging to track the tumor during treatment and can detect and compensate for tumor movement (e.g., due to breathing). No frame is bolted to the skull unlike older radiosurgery systems (like Gamma Knife®), CyberKnife® uses a soft mask or body mold instead of invasive head frames. For certain tumors (e.g., prostate, pancreas, lung), tiny fiducial markers may be implanted so the system can track them precisely. Treatments may be employed for brain tumors (benign and malignant), spinal tumors, lung, liver, pancreas, and kidney cancers, prostate cancer, and certain non-cancerous conditions (e.g., trigeminal neuralgia, arteriovenous malformations). Advantages of CyberKnife® may include non-invasive (no cutting or anesthesia), sub-millimeter accuracy, an ability to treat moving targets without needing to stop breathing or use rigid frames and an ability to be done in 1-5 treatment sessions instead of weeks.

For the purposes of the present disclosure, the term “fiducial marker” refers to a precisely placed, recognizable reference point (or set of points) used in imaging, measurement, or alignment to help systems establish position, scale, and orientation. In some disclosed embodiments, a fiducial marker is a reference point, either natural or artificial, used in imaging and other applications to help align or register images or objects. In medicine, fiducial markers are typically small metal implants, often gold, placed near a tumor to guide radiation therapy or surgery. In medical imaging and radiation therapy, the fiducial marker may help radiologists and treatment machines target the exact same spot in repeated scans or treatments.

For the purposes of the present disclosure, the term “Image-Guided Radiotherapy” (IGRT) refers to a type of radiation therapy in which medical images are taken right before or during treatment to precisely locate a tumor and adjust the radiation delivery in real time.

For the purposes of the present disclosure, the term “modality” refers to a method of treatment or a specific type of therapeutic intervention used to help patients recover from injuries, illnesses, or other conditions. It can encompass a wide range of approaches including medical imaging. Medical imaging modalities may be utilized to diagnose and monitor conditions. Examples may include, but are not limited to: X-rays: used to visualize bones and other dense tissues; CT scans: to provide detailed cross-sectional images of the body; MRI scans: using magnetic fields and radio waves to create images of soft tissues; Ultrasound: using sound waves to create images of organs and tissues; PET scans: used to visualize metabolic activity in the body.

For the purposes of the present disclosure, the term “MRI” refers to magnetic resonance imaging utilizing a medical imaging technique that uses a magnetic field and computer-generated radio waves to create detailed images of the organs and tissues in your body.

For the purposes of the present disclosure, the term “PET” refers to positron emission tomography and is a type of nuclear medicine imaging test that produces 3D images of the body's organs and tissues. It works by detecting radiation from a radioactive tracer injected into the body, showing how well organs and tissues are functioning. PET scans are particularly useful for detecting cancer, assessing its spread, and monitoring treatment effectiveness.

For the purposes of the present disclosure, the term “phantom” refers to a specially designed object that simulates human tissue or anatomy for testing, calibration, or training purposes. In medical imaging and radiation therapy, these objects may be designed with materials that mimic the physical and radiological properties of human tissues, allowing for accurate assessment of imaging performance and radiation dose delivery without exposing patients to radiation. Phantoms may vary in complexity, ranging from simple geometric shapes used for basic machine calibration to anthropomorphic models that replicate human anatomy and organ structures. Some phantoms are dynamic, capable of simulating physiological motion such as breathing, which is particularly relevant for motion management in radiotherapy. In the context of radiation therapy, phantoms may be critical for verifying dose distributions, evaluating image-guided treatment workflows, and assessing the effects of implants or fiducial markers. By providing a controlled and reproducible environment, phantoms enable optimization of treatment planning, quality assurance, and the development of new imaging or therapeutic techniques while maintaining patient safety.

For the purposes of the present disclosure, the term “proton therapy” refers to a type of radiation therapy that uses protons instead of X-rays (photons) to treat cancer. While proton therapy may still be regarded as “radiation therapy,” the physics behind protons lets doctors deliver high doses to the tumor while minimizing damage to surrounding healthy tissue.

### DESCRIPTION

While the invention is susceptible to various modifications and alternative forms, specific embodiment thereof has been shown by way of example in the drawings and will be described in detail below. It should be understood, however, that it is not intended to limit the invention to the particular forms disclosed, but on the contrary, the invention is to cover all modifications, equivalents, and alternatives falling within the spirit and the scope of the invention.

The integration of multiple radiotherapy modalities, including photon and proton therapies, within a clinical setting necessitates the use of fiducial markers that function effectively across all imaging and treatment systems. For CT, MRI, and IGRT, fiducials must be large enough to ensure visibility without introducing substantial artifacts. In CyberKnife® treatments, a fiducial diameter of at least 0.75 mm is required to maintain reliable trackability, particularly for larger patients. However, such dimensions are unsuitable for proton therapy due to significant beam perturbation. Disclosed embodiments explore the design and implementation of customized fiducials that balance visibility, trackability, and proton beam perturbation, enabling their use across multiple radiotherapy modalities.

Customized fiducials with a diameter of 0.75 mm and increased pitch values may be fabricated to reduce the amount of metal in the beam path. A phantom incorporating both standard and customized fiducials was constructed. Visibility of the fiducials may be evaluated under CT, MRI, and IGRT, with assessments conducted for both photon and proton modalities. CyberKnife® trackability is also tested. Proton beam perturbation may be quantified using Gafchromic™ EBT3 films at various distances distal to fiducials in the proton beam path.

Customized fiducials with increased pitch values may demonstrate acceptable visibility in CT, MRI, and IGRT for both photon and proton therapies. CyberKnife® trackability is largely unaffected by increases in pitch values. The customized fiducials, despite retaining a 0.75 mm diameter, significantly reduces proton beam perturbation compared to standard fiducials of the same diameter or even a smaller diameter.

Disclosed embodiments show that fiducial markers can be optimized for multi-modality compatibility by maintaining necessary dimensions for visibility and trackability while customizing pitch values to minimize proton beam perturbation. This approach simplifies treatment planning and enhances clinical efficiency by enabling the use of a single type of fiducial across multiple treatment modalities.

### Introduction

Image-guided radiotherapy (IGRT) has significantly advanced the precision of tumor targeting in radiation therapy by improving accuracy in patient positioning and target localization. Fiducial markers are crucial in IGRT, serving as reliable reference points across various treatment sites, particularly in prostate cancer radiotherapy(1-3).

In facilities offering both photon and proton therapies, the ability to use a single type of fiducial marker across multiple modalities is highly advantageous(4). However, determining the optimal fiducial for IGRT involves a careful balance between visibility and artifact production across different imaging systems(5,6). Previous studies have highlighted the importance of selecting fiducials that optimize visibility and minimize artifacts in multi-modality settings.

Proton therapy's precision is highly valued due to its conformal dose distribution and minimal exit dose(7,8). However, fiducial markers can introduce challenges in proton therapy, causing beam perturbations that lead to dosimetric uncertainties and potentially compromise treatment efficacy(9). This is particularly concerning in prostate cancer treatment, where dose perturbation can result in inadequate coverage distal to the marker. Various strategies, such as reducing the number of treatment fields, have been employed to mitigate these effects (10-14).

Recent advancements in fiducial design, such as the development of liquid markers and polymer-encapsulated markers, aim to address these challenges by reducing proton beam perturbation while maintaining visibility (15-18). Despite these efforts, the need remains for fiducials that perform effectively across all modalities (19-21). Disclosed embodiments aim to address this gap by investigating customized fiducials with increased pitch values, designed to reduce the amount of material in the proton beam path while maintaining the visibility and trackability required for CT, MRI, IGRT, and CyberKnife® treatments.

### Materials and Methods

Fiducial Fabrication: In accordance with disclosed embodiments, several fiducial options have been evaluated based on criteria including visibility, artifact production, proton beam perturbation, cost-effectiveness, and availability. The Visicoil® fiducial (IZI Medical Products) was selected for its biocompatible platinum construction, which ensures high radiographic visibility. Customized fiducials were fabricated with a diameter of 0.75 mm and varying pitch values, Table 1.

TABLE 1

| | | Diameter | Gap | Mass |
|---|---|---|---|---|
| | Type | (mm) | (mm) | (gram) |
| Fiducial 1 | Customized | 0.75 | 1.5 | 0.009 |
| Fiducial 2 | Customized | 0.75 | 1.0 | 0.0093 |
| Fiducial 3 | Customized | 0.75 | 0.25 | 0.0266 |
| Fiducial 4 | Customized | 0.75 | 1.25 | 0.008 |
| Fiducial 5 | Customized | 0.75 | 0.5 | 0.016 |
| Fiducial 6 | Customized | 0.75 | 0.75 | 0.012 |
| Fiducial 7 | Standard | 0.75 | — | — |
| Fiducial 8 | Standard | 0.50 | — | — |

These modifications were designed to reduce material in the proton beam path while maintaining adequate size for visibility and trackability across different modalities.

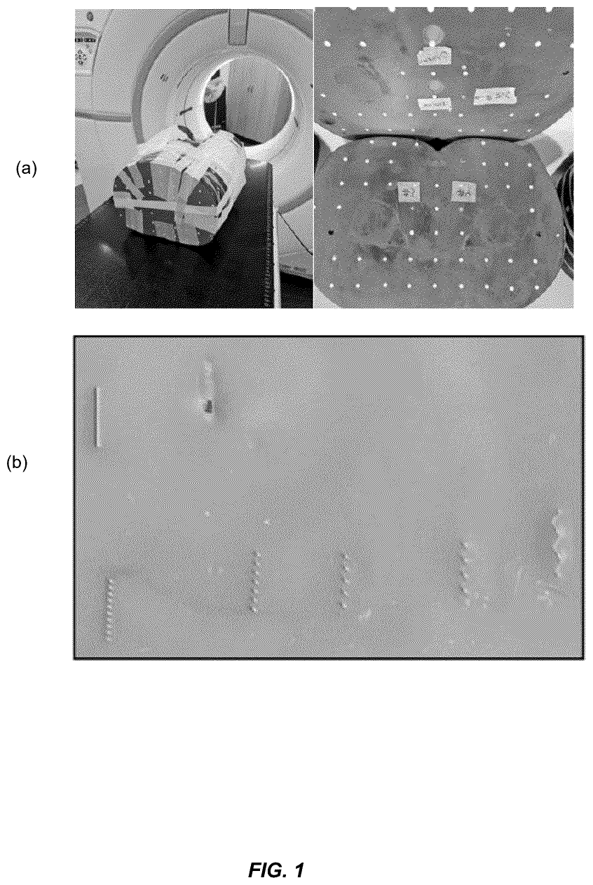

Phantom Preparation: FIG. 1. illustrates phantoms utilized for the disclosed study as follows: (a) The Rando phantom utilized for clinical imaging assessments, including visibility and trackability of fiducial markers in CT, MRI, and IGRT, as well as trackability evaluation using the CyberKnife® system. (b) The wax phantom constructed for the proton beam perturbation study, embedding both standard and customized fiducials.

Two distinct phantoms were utilized in accordance with disclosed embodiments, FIG. 1, (a), (b): Rando Phantom for Clinical Imaging Assessments: To evaluate visibility and trackability in a more clinically relevant scenario, an anthropomorphic Rando phantom was used, FIG. 1, (a). This phantom, which closely resembles the anatomical dimensions and tissue composition of a real patient, was selected to simulate realistic clinical conditions. Fiducial markers were implanted within the phantom, and imaging was performed using CT, MRI, and IGRT systems. Additionally, the trackability of the fiducials was assessed using the CK system.

Wax Phantom for Proton Beam Perturbation study: A wax phantom was constructed, embedding both standard fiducials (0.5 mm and 0.75 mm diameters) and customized fiducials (0.75 mm diameter with different pitch values), FIG. 1, (b). This phantom was used to evaluate proton beam perturbation. The setup included positioning the phantom such that the beam path was consistent across all fiducials, with variations in film optical density attributed solely to the fiducials. Gafchromic EBT3 films were placed at 14 different depths (0 to 39 mm) under the wax phantom, capturing the beam perturbation effects caused by the different fiducial designs.

Visibility and Trackability Testing: The visibility of the fiducials within the Rando phantom was assessed under photon and proton IGRT systems, as well as CT and MRI. The fiducial trackability for the CyberKnife® system was assessed with varying imaging parameters (kV and mAs). The tracking uncertainty of each fiducial given by the CyberKnife® system was used as the metric for assessing trackability.

Table 1 lists the physical characteristics of the customized fiducials. The numbering of each fiducial is consistent with the radiochromic film measurement for proton beam perturbation evaluation.

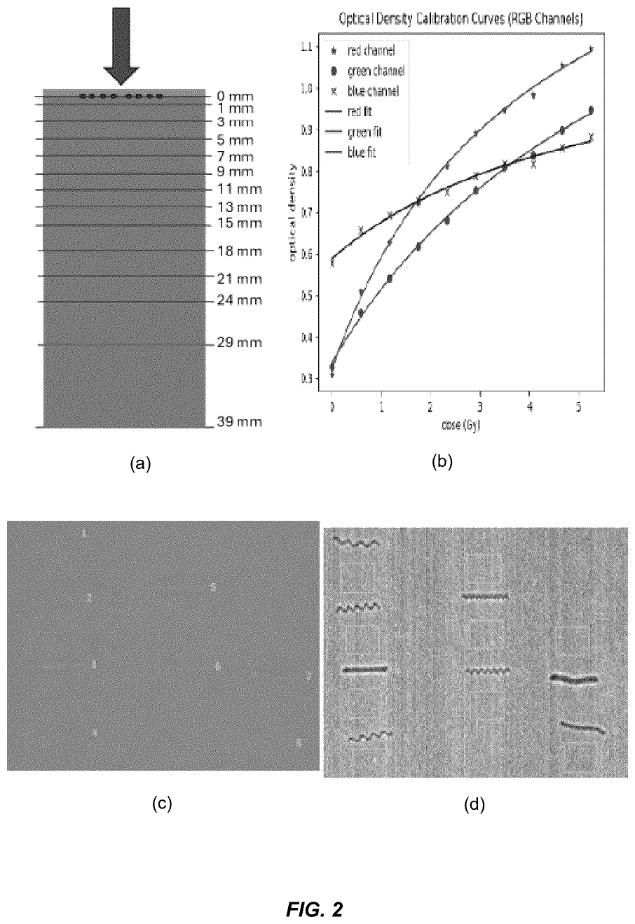

Proton Beam Perturbation Evaluation: The proton beam perturbation was quantified using the wax phantom described above. FIG. 2. illustrates disclosed proton beam perturbation evaluation as follows: (a) Schematic of the wax phantom setup, showing Gafchromic EBT3 films positioned at multiple depths (0 to 39 mm) to measure dose perturbations induced by different fiducials. (b) Film calibration curves for triple channel film dosimetry to convert optical density to dose. (c) Image of the irradiated film displaying the fiducial projection positions at depth 7 mm. (d) The corresponding dose map showing the dose decrease under the fiducial projections. The orange boxes mark the background areas that were selected to measure the average reference dose for calculating the dose perturbation. The setup ensured that any changes in film optical density were due to the fiducials, with a uniform AP SOBP proton beam covering the entire phantom.

The setup involves placing Gafchromic EBT3 films at different depths under the phantom to capture the perturbations caused by the fiducials FIG. 2, (a). An anterior-posterior (AP) spread-out Bragg peak (SOBP) proton beam, with field size large enough to cover the entire phantom, was used. This ensured that any observed changes in the films' optical densities were due to the fiducials, as the beam was forward-planned to maintain uniformity across all energy layers. To evaluate the perturbation in proton dose, the radiochromic film of the same batch was first calibrated using the three-color channels, FIG. 2, (b). The triple channel film dosimetry method (22) was used to convert the film image, FIG. 2, (c) to dose map, FIG. 2, (d).

Data Analysis: Imaging data were analyzed to assess visibility and artifact production, ensuring customized fiducials with varying pitch values provided acceptable performance across different imaging modalities. The proton beam perturbation was calculated as the relative dose change across the fiducial projection area compared to the surrounding background area for each film placed at various depths distal to the fiducials. The dosimetric impact of these perturbations was evaluated, focusing on clinically relevant dose distributions. This analysis provided insights into the extent of cold spots in target regions due to fiducials with customized pitch values compared to standard designs.

### Results

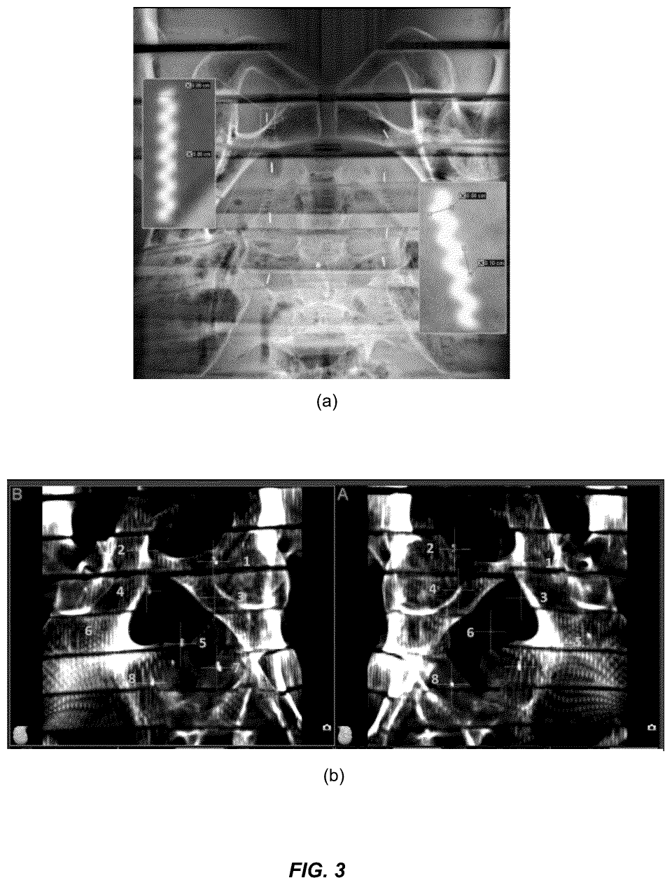

Visibility and Trackability: Customized fiducials with varying pitch values demonstrated acceptable visibility across CT, MRI, and IGRT for both photon and proton therapies. FIG. 3 illustrates a visibility assessment including: (a) CBCT image demonstrating the visibility of customized fiducials with varying pitch values, with magnified insets showing clear visibility and accurate measurements for two of them; (b) A and B, KV/KV images confirming the fiducials' visibility in IGRT. No significant imaging artifacts were observed, confirming their compatibility across modalities, FIG. 3.

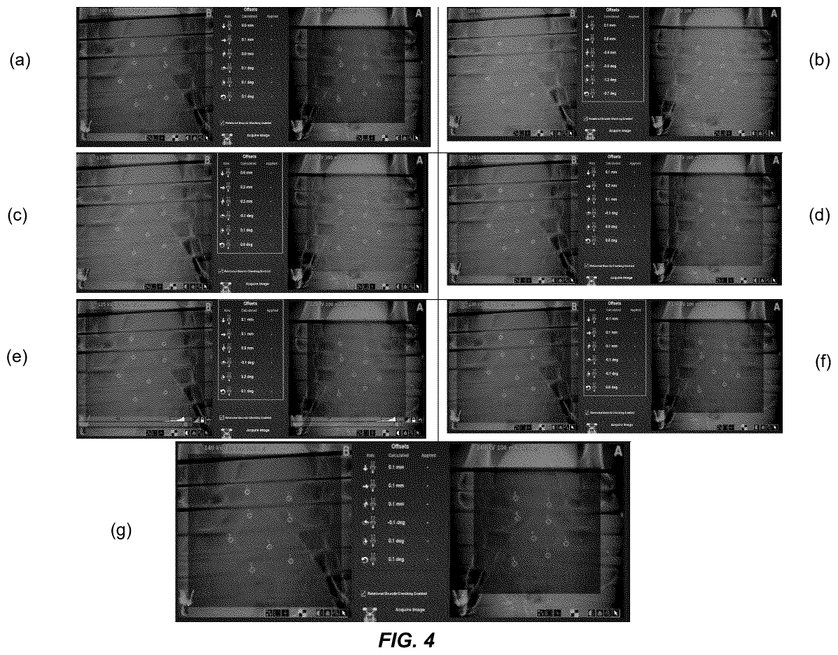

FIG. 4 illustrates fiducial tracking with a CyberKnife® System including: (a-g) Dynamic treatment simulation images showing successful tracking of each fiducial across different angles and positions, with trackability largely independent of pitch values. The CyberKnife® system successfully tracked the movement of each fiducial during dynamic treatment simulations, FIG. 4, (a-g). The trackability of the customized fiducials was largely independent of pitch values, with all fiducials maintaining reliable performance across different angles and positions.

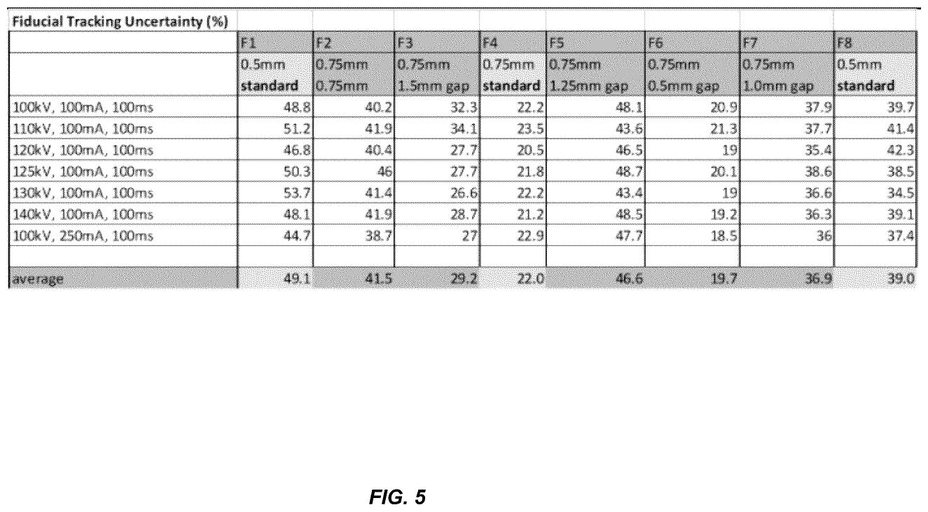

FIG. 5 is a schematic illustration showing summary of fiducial tracking uncertainties under various imaging settings, demonstrating consistent performance of fiducials across different conditions. Note that the standard fiducial with 0.5 mm diameter has been used in two different places to evaluate for the placement of the fiducial according to one embodiment of the present disclosure. In FIG. 5, different imaging settings are used to evaluate the uncertainties in tracking the fiducials.

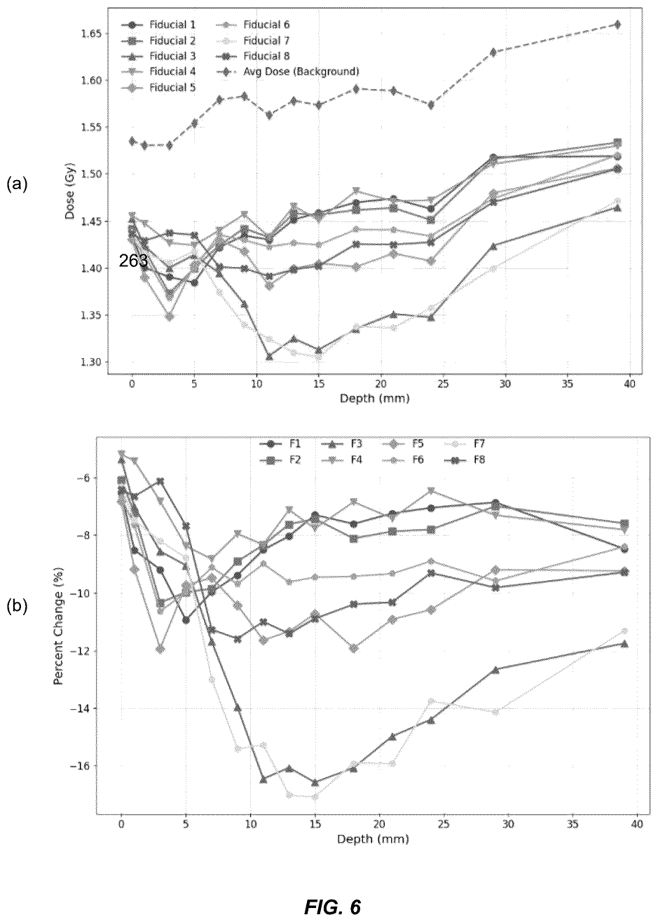

Proton Beam Perturbation: FIG. 6 graphically illustrates proton beam perturbation including: (a) the dose at each fiducial projection position (Fiducial 1-8, solid lines) as a function of depth distal to the fiducials. The average background dose at each depth is shown as the dashed line; (b) the percentage change in dose across each fiducial projection position relative to the surrounding background area as a function of depth distal to the fiducials according to one embodiment of the present disclosure. Customized fiducials demonstrated significantly reduced proton beam perturbation compared to standard 0.75 mm fiducials, FIG. 6, (a). The fiducials with varying pitch values minimized dose perturbation, leading to reduced cold spots in the target region. Comparison with non-customized fiducials, the customized fiducials outperformed standard 0.75 mm, and 0.50 mm, fiducials in minimizing proton beam perturbation while maintaining visibility and trackability FIG. 6, (b).

In some select embodiments, fiducial fabrication and phantom preparation may include selecting fiducials made of biocompatible platinum, for their high visibility and minimal proton beam perturbation. Custom fiducials with a 0.75 mm diameter and varying pitch values (0.25 mm to 1.5 mm) may be fabricated to balance visibility, trackability, and material reduction in the beam path. A wax phantom containing both standard and custom fiducials may be prepared for proton beam perturbation analysis, ensuring consistent beam paths. A simpler solid water phantom may be used for visibility and trackability tests, simulating clinical conditions.

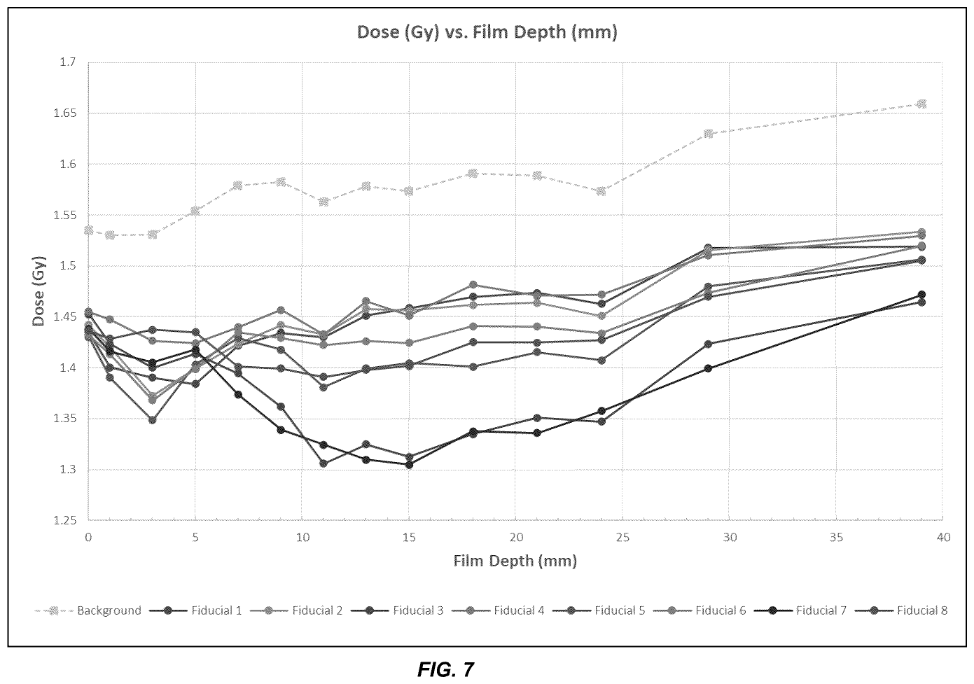

FIG. 7 graphically illustrates dose (Gy) versus film depth (mm) according to one embodiment of the present disclosure. Visibility in CT, MRI, and IGRT (Photon and Proton): customized fiducials with varying pitch values demonstrated clear visibility across photon and proton IGRT modalities, as well as in CT and MRI imaging, with no significant artifacts observed. This confirms their compatibility with multiple imaging systems, ensuring accurate target localization and patient repositioning during treatment in accordance with disclosed embodiments.

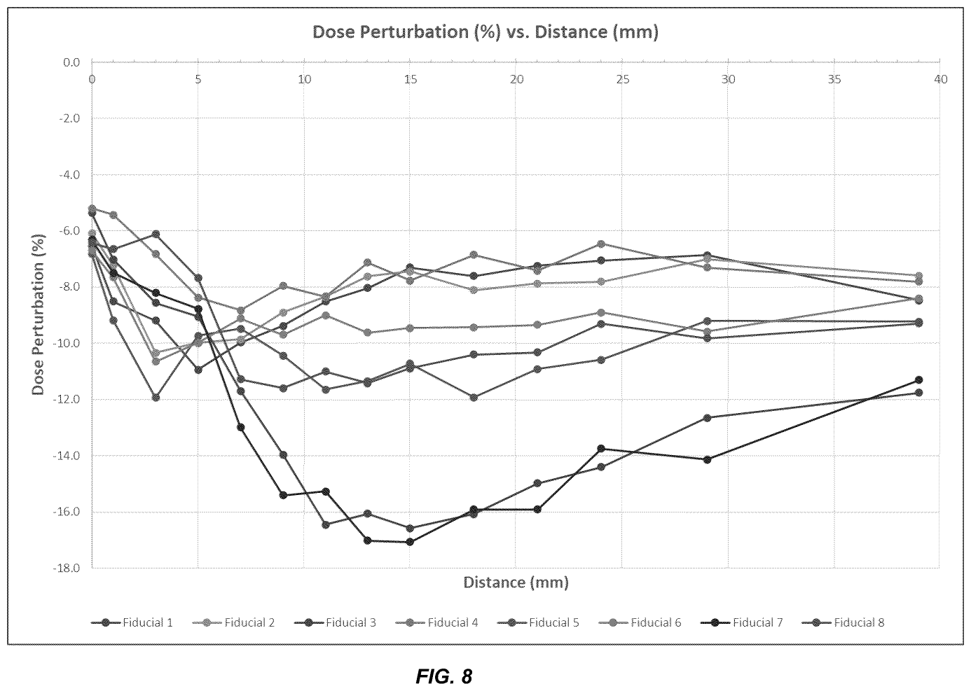

FIG. 8 graphically illustrates dose perturvation (%) versus distance (mm) according to one embodiment of the present disclosure. Trackability with CyberKnife® and proton beam perturbation: the customized fiducials maintained consistent trackability across various angles and positions using the CyberKnife® system, regardless of pitch value, ensuring reliable performance even in larger patients. In proton therapy, these fiducials significantly reduced beam perturbation compared to standard 0.75 mm fiducials. By optimizing pitch values, the fiducials minimized material in the beam path, resulting in improved dose distribution and fewer cold spots in the target regions, as demonstrated by quantitative dosimetric analysis. FIGS. 7 and 8 illustrate the impact of these fiducials on dose distribution and perturbation, highlighting their superiority over non-customized options.

Evaluation: fiducial visibility and artifact levels were tested across photon and proton IGRT systems, as well as CT and MRI imaging. Trackability was assessed using the CyberKnife® system to confirm reliability during dynamic treatments. Proton beam perturbation was evaluated using a spread-out Bragg peak (SOBP) proton beam with Gafchromic films placed at multiple depths, ensuring that any changes in film optical density were due to the fiducials, allowing for precise analysis of their impact on the beam path.

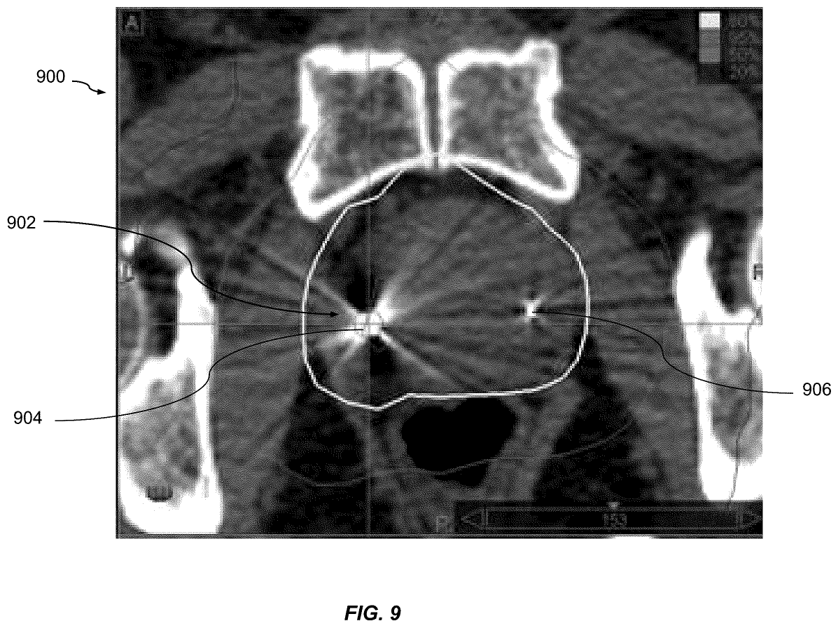

The pitch of the helix determines how tight the helix is. A fiducial marker is a balance. If the material is not dense enough it will not be easily seen on various imaging modalities. FIG. 9 is a radiological image 900 illustrating a “starburst” image 902 according to one embodiment of the present disclosure. In accordance with disclosed embodiments, if the marker is too dense, it can create a starburst artifact on X-Ray based imaging. Turning to FIG. 9, a large gold seed 904 is displayed on the left while the disclosed fiducial marker 906 is displayed on the right.

In addition, to just imaging, an advanced form of radiation therapy (proton therapy) may be employed. In proton therapy, this application uses the part of the atom that has mass wherein it is significantly imaged by the density of the fiducial markers. In FIG. 9 the imaging beam is partially blocked and scattered creating the “starburst” image. With proton therapy the same can occur, however with the actual treatment beam itself. This, in turn, may alter the treatment dose which is undesirable.

Thus, ultimately disclosed embodiments seek to create a balance between enough density to allow fiducial markers to be visible on imaging, but not too dense to impact the delivered dose of radiation. This can be achieved by using different materials of the fiducial markers, or in this instance be increasing the pitch of the helix to create a less dense coil.

Accordingly, in some disclosed embodiments, an interstitial marker configured to be imaged when inserted into soft tissue of an organ, tumor or tumor bed within a mammalian body, the marker having a longitudinal length and rings made of a non-radioactive wire, the rings being held in spaced relation along the longitudinal length of the marker, the longitudinal length of the marker is longer than an outer diameter of the rings, the marker configured for insertion into soft tissue inside the body and held by the soft tissue, the marker configured to be substantially straight just after insertion inside the soft tissue, the rings having an outer diameter of from about 25 μm to about 2500 μm, a ratio of length of the marker to outer diameter of the rings of 10 or 25 more and the rings comprising a wire having a cross section length of from about 10 μm to about 2500 μm, wherein the longitudinal length of the marker, wire cross section length, outer diameter of the rings, and ratio of longitudinal length of the marker to the outer diameter of the rings are effective to make the marker visible under at least one imaging modality selected from a group consisting of x-ray, radiography, CT scan, ultrasonography, and fluoroscopy, and wherein the longitudinal length of the marker, wire cross section length, outer diameter of the rings and ratio of longitudinal length of the marker to the outer diameter of the rings also are effective for flexibility transverse to the longitudinal length of the marker and which flexibility is effective to permit the marker to be responsive to changes of shape of the organ, tumor or tumor bed and to follow movements of the organ, tumor, or tumor bed after insertion as a substantially straight marker, but after at least one of treatment, natural growth or stresses from the soft tissue while implanted in the mammalian body; and wherein the rings are separated by a distance along the longitudinal length of the marker, and a ratio of wire cross section length to the separation distance of the rings is about 1:1.2±about 0.2, the separation distance, the marker longitudinal length, the wire cross section length, the outer diameter of the rings, the ratio of longitudinal length to outer diameter of the rings effective for providing lateral flexibility transverse to the longitudinal length of the marker. Furthermore, in some disclose embodiments, a portion of the flexible wire may be in the form of a helical coil, wherein the portion comprises sections of different pitch. Portions of the marker may be made of radiopaque material. The marker may include one or more tissue anchors which attach the marker to the organ, tumor or tumor bed. In some embodiments, the marker is made of biocompatible material suitable for permanent implantation selected from a group, for example, consisting of rhodium, platinum, iridium, tantalum, titanium, silver, gold, nickel, alloys containing these metals, and stainless steel. Furthermore, some embodiments may provide the wire having a circular cross section with a diameter of from about 10 μm to about 2500 μm, or a rectangular cross section with a length of from about 10 μm to about 500 μm.

Thus, in some exemplary embodiments, the disclosed pitch may be regarded as a ratio of coil length to coil diameter. And in some primary embodiments, the pitch may have a range of about 0.25 to about 1.5 mm. Additionally, some disclosed criteria may include a substantially zero pitch or gap within the helix or coil. In other embodiments, the criteria may relate to the overall coil (helix) length, which may range from about 3 mm to about 10 mm.

### Discussion

Disclosed embodiments demonstrate the feasibility of optimizing fiducials for use across multiple radiotherapy modalities. By customizing the pitch values of fiducials, disclosed embodiments are able to reduce proton beam perturbation while maintaining the necessary dimensions for visibility and trackability in CT, MRI, IGRT, and CyberKnife® systems. These findings have significant clinical implications, particularly in settings where multiple treatment modalities are available and the final treatment approach may not be determined at the time of fiducial implantation.

The ability to use a single fiducial type across different modalities simplifies the treatment planning process and enhances clinical efficiency. This is especially valuable in proton therapy, where minimizing beam perturbation is critical for maintaining dose integrity and ensuring effective treatment. Further clinical validation is required to confirm these findings in a broader range of clinical scenarios. Long-term studies on the stability and performance of customized fiducials in vivo will provide additional insights into their clinical utility.

### CONCLUSION

Disclosed embodiments demonstrate that fiducials can be optimized for compatibility across multiple radiation treatment modalities. By customizing pitch values, we achieved acceptable visibility and trackability for CyberKnife® while minimizing proton beam perturbation. The use of customized fiducials enhances the flexibility of treatment planning and improves clinical outcomes by ensuring precise dose delivery. Future research should focus on long-term clinical validation and potential improvements in fiducial design to further enhance their performance in IGRT.

### REFERENCES

The following references are referred to above and are incorporated herein by reference:
1. Dang A, Kupelian P A, Cao M, Agazaryan N, Kishan A U, Image-guided radiotherapy for prostate cancer. Transl Androl Urol. 2018 June; 7(3):308-20.
2. Chan M F, Cohen G N, Deasy J O. Qualitative Evaluation of Fiducial Markers for Radiotherapy Imaging. Technol Cancer Res Treat. 2015 June; 14(3):298-304.
3. O'Neill A G M, Jam S., Hounsell A R, O'Sullivan J M. Fiducial marker guided prostate radiotherapy: a review. The British Journal of Radiology. 2016 Dec. 1; 89(1068):20160296.
4. Handsfield L L, Yue N J, Zhou J, Chen T, Goyal S. Determination of optimal fiducial marker across image-guided radiation therapy (IGRT) modalities: visibility and artifact analysis of gold, carbon, and polymer fiducial markers. J Applied Clin Med Phys. 2012 September; 13(5):181-9.
5. Chen Y, J O'Connell J, Ko C J, Mayer R R, Belard A, McDonough J E. Fiducial markers in prostate for kV imaging: quantification of visibility and optimization of imaging conditions, Phys Med Biol. 2012 Jan. 7; 57(1):155-72.
6. Osman S O S, Russell E, King R B, Crowther K, Jain S, McGrath C, et al. Fiducial markers visibility and artefacts in prostate cancer radiotherapy multi-modality imaging. Radiat Oncol. 2019 December; 14(1):237
7. Newhauser W D, Zhang R. The physics of proton therapy. Phys Med Biol. 2015 Apr. 21; 60(8):R155-209.
8. Lane S A, Slater J M, Yang G Y. Image-Guided Proton Therapy: A Comprehensive Review. Cancers, 2023 Apr. 29; 159):2555,
9. Parhama M D, Ahrnad S, Jin H. Dosimetric Effect of Biozorb Markers for Accelerated Partial Breast Irradiation in Proton Therapy. International Journal of Particle Therapy. 2021 March; 7(4):19-28,
10. Lim Y K, Kwak J, Kim D W, Shin D, Yoon M, Park S, et al. Microscopic Gold Particle-Based Fiducial Markers for Proton Therapy of Prostate Cancer. International Journal of Radiation Oncology*Biology*Physics. 2009 August; 74(5):1609-16.
11. Giebeler A. Fontenot J. Baiter P, Ciangaru G, Zhu R, Newhauser W. Dose perturvations from implanted helical gold markers in proton therapy of prostate cancer. J Applied Clin Med Phys. 2009 December; 10(1):63-70.
12. Huang J Y, Newhauser W D, Zhu X R, Lee A K, Kudchadker R J. Investigation of dose perturbations and the radiographic visibility of potential fiducials for proton radiation therapy of the prostate. Phys Med Biol. 2011 Aug. 21; 56(16):5287-302.
13. Matsuura T, Maeda K, Sutherland K, Takayanagi T, Shimizu S, Takao S, et al. Biological effect of dose distortion by fiducial markers in spot-scanning proton therapy with a limited number of fields: A simulation study. Medical Physics. 2012 September; 39(9):5584-91.
14. Kudchadker R J, Lee A K, Yu Z H, Johnson J L, Zhang L, Zhang Y, et al. Effectiveness of Using Fewer Implanted Fiducial Markers for Prostate Target Alignment. International Journal of Radiation Oncology*Biology*Physics. 2009 July; 74(4):1283-9.
15. Cheung J, Kudchadker R J, Zhu X R, Lee A K, Newhauser W D. Dose perturbations and image artifacts caused by carbon-coated ceramic and stainless steel fiducials used in proton therapy for prostate cancer. Phys Med Biol. 2010 Dec. 7; 55(23):7135-47.
16. Scherman Rydhög J, Perrin R, Jølck RI, Gagnon-Moisan F, Larsen K R, Clementsen P, et al, Liquid fiducial marker applicability in proton therapy of locally advanced lung cancer. Radiotherapy and Oncology. 2017 March; 122(3):393-9.
17. Wang L, Sanders J, Ward J F. Lee S R, Poenisch F, Swanson D M, et al. A Novel Polymer-Encapsulated Multi-Imaging Modality Fiducial Marker with Positive Signal Contrast for Image-Guided Radiation Therapy. Cancers. 2024 Jan. 31; 16(3):625.
18. Zhang M. Reyhan M, Kim L H. Depth dose perturbation by a hydrogel fiducial marker in a proton beam. J Applied Clin Med Phys. 2015 January; 16(1):373-6.
19. Reidel C A, Horst F, Schuy C, Jäkel O, Ecker S, Henkner K, et al. Experimental Comparison of Fiducial Markers Used in Proton Therapy: Study of Different Imaging Modalities and Proton Fluence Perturbations Measured With CMOS Pixel Sensors. Front Oncol. 2022 Mar. 25; 12:830080.
20. Lim Joon D, Berry C, Harris B, Tacey M, Smith D. Lawrentschuk N, et al. A clinical study comparing polymer and gold fiducials for prostate cancer radiotherapy. Front Oncol. 2023 Feb. 1; 12:1023288.
21. Lim Joon D, Smith D, Tacey M, Schneider M, Harris B, Ong W L, et al. A phantom study to contrast and compare polymer and gold fiducial markers in radiotherapy simulation imaging. Sci Rep. 2021 Apr. 26; 11(1):8931.
22. Andre Micke, David F. Lewis, Xiang Yu, Multichannel film dosimetry with nonuniformity correction. Medical Physics. 2011 May; 38(5): 2523-34.

All documents, patents, journal articles and other materials cited in the present application are incorporated herein by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

What is claimed is:

1. An interstitial marker configured to be imaged when inserted into soft tissue of a subject's body,
wherein the interstitial marker is optimized for multi-modality compatibility by maintaining necessary dimensions for visibility, trackability and material reduction in a beam path via customized pitch values to minimize proton beam perturbation, having a pitch value in a range of about 0.25 mm to about 1.5 mm, and
wherein the interstitial marker has a diameter of at least 0.50 mm.

2. The interstitial marker of claim 1, wherein the interstitial marker is a fiducial marker.

3. The interstitial marker of claim 1, wherein the interstitial marker has a diameter in a range of about 0.50 mm to about 0.75 mm

4. The interstitial marker of claim 1, wherein the interstitial marker has a mass in the range of 0.009 gram to 0.0266 gram.

5. The interstitial marker of claim 1, wherein the interstitial marker is configured to perform effectively across multiple treatment modalities.

6. The interstitial marker of claim 1, wherein the visibility and trackability of the interstitial marker is capable of evaluation under CT, MRI, IGRT, and CyberKnife® systems.

7. The interstitial marker of claim 1, wherein the interstitial marker is capable of providing assessments conducted for both photon and proton modalities.

8. The interstitial marker of claim 1, wherein the interstitial marker has a longitudinal length and rings made of a non-radioactive wire, the rings being held in spaced relation along the longitudinal length of the marker.

9. The interstitial marker of claim 8, wherein the longitudinal length of the interstitial marker is longer than an outer diameter of the rings.

10. The interstitial marker of claim 9, wherein the rings have an outer diameter of from 25 μm to 2500 μm.

11. The interstitial marker of claim 8, wherein a ratio of length of the interstitial marker to outer diameter of the rings is 10 or 25 more.

12. The interstitial marker of claim 8, wherein the a wire has a cross section length of from 10 μm to 2500 μm.

13. The interstitial marker of claim 8, wherein the interstitial marker is configured for insertion into soft tissue inside the subject's body and held by the soft tissue.

14. The interstitial marker of claim 8, wherein the longitudinal length of the interstitial marker, wire cross section length, outer diameter of the rings and ratio of longitudinal length of the interstitial marker to the outer diameter of the rings are effective for flexibility transverse to the longitudinal length of the marker and which flexibility is effective to permit the interstitial marker to be responsive to changes of shape of an organ, tumor or tumor bed and to follow movements of the organ, tumor, or tumor bed after insertion.

15. The interstitial marker of claim 8, wherein the rings are separated by a distance along the longitudinal length of the interstitial marker, and a ratio of wire cross section length to a separation distance of the rings is about 1:1.2±about 0.2.

16. The interstitial marker of claim 15, wherein the separation distance, the marker longitudinal length, the wire cross section length, the outer diameter of the rings, the ratio of longitudinal length to outer diameter of the rings is effective for providing lateral flexibility transverse to the longitudinal length of the marker.

17. The interstitial marker of claim 8, wherein the non-radioactive wire is flexible wire, wherein a portion of the flexible wire are formed in a helical coil, wherein the portion comprises sections of different pitch.

18. The interstitial marker of claim 8, wherein the non-radioactive wire has a circular cross section with a diameter of from 10 μm to 2500 μm, or a rectangular cross section with a length of from 10 μm to 500 μm.

19. The interstitial marker of claim 1, wherein portions of the interstitial marker are made of radiopaque material.

20. The interstitial marker of claim 1, wherein the interstitial marker is made of biocompatible material suitable for permanent implantation selected from at least one member of a group consisting of rhodium, platinum, iridium, tantalum, titanium, silver, gold, nickel, and stainless steel, or mixtures thereof.
